(19) United States
(12) Patent Application Publication
(10) Pub. No.: US 2013/0204064 A1
(43) Pub. Date: Aug. 8, 2013

(54) Method of Making Supported Copper Adsorbents Having Copper at Selectively Determined Oxidation Levels

(71) Applicants: Vladislav Ivanov Kanazirev, Arlington Heights, IL (US); Stephen R. Caskey, Franklin, WI (US)

(72) Inventors: Vladislav Ivanov Kanazirev, Arlington Heights, IL (US); Stephen R. Caskey, Franklin, WI (US)

(73) Assignee: UOP LLC, Des Plaines, IL (US)

(21) Appl. No.: 13/367,348

(22) Filed: Feb. 6, 2012

(51) Int. Cl. C07C 7/12

(52) U.S. Cl. 585/823

(57) ABSTRACT

A method of removing O₂, CO, H₂, mercury, and/or sulfur from a fluid stream using a sorbent comprising metallic copper. The metallic copper is formed from direct reduction of a supported copper oxysalt by exposure to a reducing agent at a temperature of between about 40° C. and about 220° C.

### FIELD OF THE INVENTION

The disclosure relates in general to the removal of contaminants from hydrocarbon liquids and gases. In certain embodiments, the disclosure relates to the use of a copper-based sorbent to remove contaminants from hydrocarbon streams. In certain embodiments, the disclosure relates to the use of a sorbent comprising metallic copper, where the metallic copper was produced by way of direct reduction of a copper oxysalt.

### BACKGROUND OF THE INVENTION

Copper-containing sorbents are often used to scavenge contaminants from fluid (i.e., gas or liquid) streams. The active component of the sorbent is often a copper compound at a particular level of oxidation. The level of oxidation is selected based on the particular contaminants in the fluid stream and on various operating conditions. For example, sorbents containing copper (II), copper at a +2 oxidation state, in the form of cupric oxide (CuO) are highly effective for sulfur and mercury scavenging. Sorbents containing copper (I), copper at a +1 oxidation state, in the form of cuprous oxide (Cu₂O) are highly effective for contaminant removal at elevated temperatures. And finally, sorbents containing metallic copper (Cu), copper at a +0 oxidation state, are highly effective for O₂, CO, and H₂ removal.

Prior art processes include a first step of thermally decomposing a copper carbonate, such as Cu—Zn carbonate, by exposure to heat to produce supported cupric oxide (CuO). In a second step, the cupric oxide, containing copper at a +2 oxidation state, is then reduced at a relatively high temperature to produce supported metallic copper (Cu).

The Hüttig and Tamman temperatures of a material indicate the temperatures at which sintering (or agglomeration) of the material may occur and are related to the melting temperature. As the temperature of the material increases, the mobility of the atoms in the material increases. At the Hüttig temperature, atoms at crystalline defects within the material will begin exhibiting mobility. At the Tamman

temperature, atoms within the bulk material begin exhibiting mobility. At the melting point of the material, the mobility of the atoms within the material is an high that liquid-phase behavior is observed. The semi-empirical approximation for the Tamman and Hüttig temperatures, in kelvins, is shown in (1) and (2).

T_{Hüttig}(K)−0.3*T_{melting}(K) (1)

T_{Tamman}(K)=0.5*T_{melting}(K) (2)

Additional discussion of the Hüttig and Tamman temperatures can be found in J. Moulijn, Applied Catalysis A: General 212, 9-10 (2001), which provides the specific values of the Hüttig and Tamman temperatures for metallic copper, cupric oxide, and cuprous oxide as listed in Table 1.

TABLE 1

| Material | T_{Melting} | T_{Hüttig} | T_{Tamman} |
|---|---|---|---|
| Metallic Copper (Cu) | 1083° C. | 405° C. | 134° C. |
| Cupric Oxide (CuO) | 1326° C. | 527° C. | 207° C. |
| Cuprous Oxide (Cu₂O) | 1235° C. | 481° C. | 179° C. |

The actual Hüttig and Tamman temperatures for a copper-based material, however, will vary from the numbers in Table 1 based on several factors, such as texture, size and morphology of the material.

The temperature necessary to reduce the cupric oxide to metallic copper is generally above the Hüttig and/or Tamman temperatures of these materials. It is most desirable for the active copper component of the sorbent to have a high surface area, and therefore a small crystalline size, to increase the amount of copper available for scavenging reactions. As such, the agglomeration of copper during the formation of metallic copper in the sorbent is undesirable because agglomeration results in larger copper particle sizes, less available surface area, and less effective sorbent performance.

Accordingly, it would be an advance in the state of the art to provide a method of producing copper-based sorbents that (i) avoid agglomeration of the metallic copper component by remaining below the Hüttig and Tamman temperatures during formation of metallic copper, (ii) consume less energy during production, and (iii) permit the formation of sorbents comprising copper at one or more levels of oxidation and in varying amounts at each level of oxidation so as to produce sorbents targeted to a specific application.

### SUMMARY OF THE INVENTION

A method of removing from a fluid stream at least one impurity selected from the group consisting of O₂, CO, H₂, mercury, and sulfur. The method contacts the stream with a sorbent comprising metallic copper. The metallic copper is formed from direct reduction of a supported copper oxysalt by exposure to a reducing agent at a temperature of between about 40° C. and about 220° C.

### DETAILED DESCRIPTION OF PREFERRED EMBODIMENTS

The invention is described in preferred embodiments in the following description. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The terms sorbent, adsorbent, and absorbent as used herein refer to the ability of a material to take in or soak up liquid or gas components on the surface thereof or to assimilate such components into the body thereof.

Methods of producing copper-based sorbents, and sorbents produced by such methods, are presented. In one embodiment, Applicants' sorbent comprises a copper material disposed within a support material. In various embodiments, the sorbent comprises a copper material and a reduction inhibitor, such as a halide salt, disposed within a support material. In various embodiments, the sorbent comprises a copper oxide disposed within a support material. In various embodiments, the copper material is a copper compound with copper at a +2 oxidation state, a copper compound with copper at a +1 oxidation state, a copper compound with copper at a +0 oxidation state, or a combination thereof. In one embodiment, the copper at a +2 oxidation state is cupric oxide (CuO). In one embodiment, the copper at a +1 oxidation state is cuprous oxide (Cu₂O). The copper at a +0 oxidation state is metallic copper.

In various embodiments, the support material is a metal oxide selected from the group consisting of alumina, silica, silica-aluminas, silicates, aluminates, silico-aluminates such as zeolites, titania, zirconia, hematite, ceria, magnesium oxide, and tungsten oxide. In one embodiment, the support material is alumina. In some embodiments, the support material is carbon or activated carbon. In certain embodiments, Applicants' sorbent does not comprise a binder.

In various embodiments, the alumina support material is present in the form of transition alumina, which comprises a mixture of poorly crystalline alumina phases such as “rho,” “chi” and “pseudo gamma” aluminas which are capable of quick rehydration and can retain substantial amounts of water in a reactive form. An aluminum hydroxide Al(OH)₃, such as gibbsite, is a source for preparation of transition alumina. The prior art industrial process for production of transition alumina includes milling gibbsite to 1-20 microns particle size followed by flash calcination for a short contact time as described in the patent literature such as in U.S. Pat. No. 2,915,365. Amorphous aluminum hydroxide and other naturally found mineral crystalline hydroxides e.g., Bayerite and Nordstrandite or monoxide hydroxides, AlOOH, such as Boehmite and Diaspore can be also used as a source of transition alumina. In one embodiment, the BET surface area of this transition alumina material is about 300 m²/g and the average pore diameter is about 30 angstroms as determined by nitrogen adsorption.

In various embodiments, a solid oxysalt of a transitional metal is used as a starting component of the sorbent. “Oxysalt,” by definition, refers to any salt of an oxyacid. Sometimes this definition is broadened to “a salt containing oxygen as well as a given anion.” FeOCl, for example, is regarded as an oxysalt according this definition.

In certain embodiments, the oxysalt comprises one or more copper carbonates. Basic copper carbonates can be produced by precipitation of copper salts, such as Cu(NO)₃, CuSO₄ and CuCl₂, with sodium carbonate. In one embodiment, the oxysalt is a synthetic form of malachite, a basic copper carbonate, produced by Phibro Tech, Ridgefield Park, N.J. In one embodiment, the oxysalt is a basic copper carbonate with the formula Cu₂CO₃(OH)₂. In one embodiment, the oxysalt comprises mixed copper carbonates, such as, without limitation, a mixture of CuCO₃(OH)₂ and Cu₂CO₃(OH)₂.

Depending on the conditions used, and especially on washing the resulting precipitate, the final material may contain some residual product from the precipitation process. In the case of the CuCl₂ raw material, sodium chloride is a side product of the precipitation process. It has been determined that a commercially available basic copper carbonate that had both residual chloride and sodium, exhibited lower stability towards heating and improved resistance towards reduction than other commercial basic copper carbonates that were practically chloride-free.

In one embodiment, the particle size of the basic copper carbonate particles is approximately in the range of that of the transition alumina, namely 1-20 microns. In other embodiments, the sorbent comprises the oxysalt Azurite, Cu₃(CO₃)₂(OH)₂. In other embodiments, the sorbent comprises an oxysalt of copper, nickel, iron, manganese, cobalt, zinc or a mixture thereof.

In certain embodiments, the sorbent is produced by calcinating a mixture of an inorganic halide additive and basic copper carbonate for a sufficient period of time to thermally decompose the basic copper carbonate into an oxide. In various embodiments, the inorganic halides are sodium chloride, potassium chloride or mixtures thereof In certain embodiments, the inorganic halides are bromide salts. In various embodiments, the chloride content in the sorbent ranges from 0.05 to 2.5 mass percent. In various embodiments, the chloride content in the sorbent ranges from 0.3 to 1.2 mass percent. The copper oxide-based sorbent that contains the halide salt exhibits a higher resistance to reduction than does a similar sorbent that is made without the halide salt. In certain embodiments, the preferred halide is chloride.

In various embodiments, and depending on the application, the sorbent comprises about 5 mass percent copper to about 95 mass percent copper, calculated as CuO on a volatile-free basis. In various embodiments, and depending on the application, the sorbent comprises between about 25 mass percent copper and about 50 mass percent copper, calculated as CuO on a volatile-free basis. In one embodiment, the sorbent comprises about 32 mass percent copper calculated as CuO on a volatile-free basis. In one embodiment, the sorbent comprises about 68 mass percent copper calculated as CuO on a volatile-free basis.

In one embodiment, the sorbent is produced by conodulizing basic copper carbonate with alumina followed by curing and activation. In various embodiments, the nodulizing, or agglomeration, is performed in a pan or a drum. The materials are agitated by the oscillating or rotating motion of the nodulizer or agglomerizer while spraying with water to form beads, which may be spherical or irregularly shaped. In other embodiments, the beads are formed by extrusion.

In one embodiment, sodium chloride is added to the water to form an about 1% to about 3% solution. In one embodiment, the beads are cured at about 60° C. and dried in a moving bed activator at a temperature at or below about 175° C. In one embodiment, the sorbent beads comprise between about 0.5 mass percent and about 0.8 mass percent chloride in the final dried product.

In certain embodiments, substantially all of the copper carbonate in the sorbent beads is reduced by exposure to a reducing agent at a temperature below 250° C. In certain embodiments, the reduction occurs below about 200° C. In certain embodiments, the reduction occurs below about 150° C. In one embodiment, the reduction occurs at about 200° C. In one embodiment, the reduction occurs at about 130° C.

In one embodiment, the reducing agent is hydrogen gas (H₂). In other embodiments, reducing agents other than hydrogen is used, such as natural gas or methane gas (CH₄). In one embodiment, the copper carbonate is reduced by exposing the beads to a mixture of 5% hydrogen in helium at a temperature of 220° C. In one embodiment, the copper carbonate is directly reduced to metallic copper without first being thermally decomposed into an intermediate oxide by reaction (3).

Cu₂(OH)₂CO₃+2H₂→2Cu+3H₂O+CO₂ (3)

In one embodiment, copper in the copper carbonate is directly reduced to cuprous oxide (Cu₂O) by reaction (4).

Cu₂(OH)₂CO₃+H₂→Cu₂O2H₂O+CO₂ (4)

In certain embodiments, a portion of the copper carbonate in the sorbent beads is directly reduced to metallic copper and another portion is directly reduced to copper oxide by exposure to a reducing agent at temperatures below about 250° C. In certain embodiments, the reduction occurs below about 200° C. In certain embodiments, the reduction occurs below about 150° C. In one embodiment, the reduction occurs at about 130° C. In various embodiments, only a portion of the copper carbonate is reduced to metallic copper by reaction (3) or to cuprous oxide by reaction (4) while another portion is thermally decomposed to cupric oxide (CuO) by reaction (5).

Cu₂(OH)₂CO₃→2CuO+H₂O+CO₂ (5)

The decomposition of copper carbonate generally occurs at or greater than about 290° C. In a mixed environment of helium (He) and hydrogen (H₂), decomposition of copper carbonate occurs at a much lower temperature, 220° C., and is accompanied by reduction. As such, reduction to cuprous oxide (Cu₂O) and metallic copper (Cu) occurs simultaneously. In one embodiment, the ratio of hydrogen to helium is about 5 volume percent/95 volume percent. In some embodiments, the ratio of hydrogen to helium is about 1 volume percent/40 volume percent. Applicants' method therefore involves a single active processing step for producing adsorbents comprising metallic copper and at a much lower temperature than the two-step decomposition-reduction process of the prior art.

In certain embodiments, the reduction occurs by exposing the sorbent beads to a atmosphere comprising a reducing agent. In one embodiment, the reducing agent comprises hydrogen at a partial pressure of between about 0.5 bar (7 psi) to about 120 bar (1740 psi). In certain embodiments, the atmosphere comprises a flowing hydrogen stream. In certain embodiments, the copper in the sorbent beads is directly reduced in an atmosphere comprising hydrogen at a high partial pressure at temperatures between about 40° C. and about 130° C. As the partial pressure of hydrogen increases, the temperature necessary for reduction decreases.

In one embodiment, the copper in the sorbent beads is directly reduced in a high pressure flowing hydrogen environment at a temperature of about 40° C. In one embodiment, the copper in the sorbent beads is directly reduced in a high pressure flowing hydrogen environment at a temperature of about 50° C. In certain embodiments, the partial pressure of the hydrogen is between about 10 (145 psi) bar and about 120 bar (1740 psi). In certain embodiments, the sorbent bead is directly reduced at a temperature between about 40° C. and about 220° C. with a reducing agent in an environment comprising a hydrogen partial pressure between about 0.2 bar (3 psi) and about 120 bar (1740 psi). In certain embodiments, the sorbent bead is directly reduced at a temperature between about 40° C. and about 105° C. with a reducing agent in an environment comprising a hydrogen partial pressure between about 10 bar (145 psi) and about 120 bar (1740 psi) for between about 3 hours and about 120 hours.

In various embodiments, the reduction occurs in an atmosphere comprising a reduction agent, such as without limitation hydrogen, carbon monoxide (CO), synthesis gas (a gas mixture comprising various amounts of carbon monoxide and hydrogen), hydrocarbons (including without limitation methane), or a combination thereof.

In another embodiment, a portion of the copper carbonate is directly reduced to metallic copper by reaction (1), another portion is directly reduced to cuprous oxide (Cu₂O) by reaction (2), and yet another portion is decomposed to cupric oxide (CuO) by reaction (3).

In one embodiment, substantially all the copper in the copper carbonate is decomposed and/or reduced to form Cu, CuO, and Cu₂O. In one embodiment, the sorbent comprises a halide ion reduction inhibitor, such as chloride ions, to increase the resistance to reduction. As such, the respective amounts of Cu, CuO, and Cu₂O in the final sorbent product can be varied by varying the amount of chloride in the sorbent. The reduction reaction predominates in a sorbent without chloride, resulting in a final product where substantially all the copper is fully reduced to metallic copper (i.e., the sorbent comprises no copper oxide, such as cupric oxide and/or cuprous oxide). In comparison, the decomposition reaction predominates in a sorbent with a high amount of chloride, resulting in a final product where substantially all copper is decomposed to cupric oxide (CuO). In some embodiments, as would be appreciated to those skilled in the art, the length of heating for decomposition, choice of reduction agent, pressure of the atmosphere in which reduction occurs, length of exposure to reduction agents, amount of chloride, or a combination thereof are used to selectively determine the ratio of Cu/CuO/Cu₂O. The ratio of Cu/CuO/Cu₂O in the final sorbent product is determined based on a particular application. In one embodiment, the ratio of Cu/CuO/Cu₂O is about 10%/85%/5%. In another embodiment, the ratio of Cu/CuO/Cu₂O is about 50%/5%/45%.

The Hüttig temperature of metallic copper is approximately 134° C. and the Tamman temperature of metallic copper is approximately 405° C. Unlike reduction of cupric oxide (CuO) to form metallic copper, the reduction and decomposition of copper carbonate as described in the preceding paragraph occurs below both the Hüttig and Tamman temperatures. As such, agglomeration of the active metallic copper component of the sorbent is minimized over prior art methods.

The following Example is presented to further illustrate to persons skilled in the art how to make and use the invention. This Example is not intended as a limitation, however, upon the scope of Applicants' invention.

### EXAMPLE

A mixture of a copper oxysalt and a support material is provided. The copper oxysalt is basic copper carbonate, Cu₂(OH)₂CO₃ and the support material is alumina powder capable of rehydration. In different embodiments, the copper content of the mixture, calculated as CuO on a volatile-free basis, is between about 5% and about 95%. In certain embodiments, the copper content of the mixture, calculated as CuO on a volatile-free basis, is between about 25% and about 50%. In one embodiment, the copper content of the mixture is about 32%. In one embodiment, the copper content of the mixture is about 68%.

Green sorbent beads are then formed from the mixture. As used herein, “green sorbent beads” refer to beads containing the copper oxysalt before any decomposition or reduction and “activated sorbent beads” refer to beads where at least a portion of the copper oxysalt has been decomposed or reduced. In one embodiment, the beads are formed by nodulizing the mixture in a rotating pan nodulizer while spraying with a liquid. In one embodiment, the liquid comprises water. In one embodiment, the liquid comprises a solution of water and a halide salt. In one embodiment, the halide salt is sodium chloride. The amount of sodium chloride in solution is selected based on the desired ratio of the various active copper components in the final product (i.e., Cu, CuO, and/or Cu₂O). In one embodiment, the solution comprises between about 1 mass percent and about 3 mass percent solution of sodium chloride.

In another embodiment, the green sorbent beads are formed by agglomeration. In another embodiment, the green sorbent beads are formed by extrusion. Those skilled in the art will appreciate that other methods may be performed to produce regular- or irregular-shaped beads, with or without a halide salt, that fall within the scope of Applicants' invention.

The green sorbent beads are cured and dried. In one embodiment, the curing occurs at about 60° C. In one embodiment, the beads are dried in a moving bed activator at temperatures at or below 175° C. In one embodiment, the activated sorbent beads comprise about 0.5 mass percent to about 0.8 mass percent chloride.

The green sorbent beads are activated by exposure to a reducing agent at a temperature below the Hüttig temperature of the final active copper component(s) in the sorbent. The length of exposure, the composition of the reducing agent, and temperature are selected based on the desired composition of the active copper components in the final sorbent product. In one embodiment, the reducing agent comprises about 5% hydrogen (H₂) in helium at about 220° C. for about 10 minutes. In one embodiment, the activated sorbent bead comprises a ratio of Cu/CuO/Cu₂O of about 50%/5%/45%. In another embodiment, the activated sorbent bead comprises a ratio of Cu/CuO/Cu₂O of about 10%/85%/5%. In one embodiment, the metallic copper in the activated sorbent bead comprises at least 10 mass percent of the copper-containing material in the bead.

The activated beads are then placed in a hydrocarbon stream to scavenge impurities. In various embodiments, the impurities are O₂, CO, H₂, mercury (including mercury-containing compounds), sulfur (including sulfur-containing compounds), or a combination thereof.

The described features, structures, or characteristics of the invention may be combined in any suitable manner in one or more embodiments. In the above description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention. In other words, the present invention may be embodied in other specific forms without departing from its spirit or essential characteristics. The described implementations are to be considered in all respects only as illustrative and not restrictive. The scope of the invention should, therefore, be determined not with reference to the above description, but instead should be determined with reference to the pending claims along with their full scope or equivalents, and all changes which come within the meaning and range of equivalency of the claims are to be embraced within their full scope.

1. A method of removing from a fluid stream at least one impurity selected from the group consisting of O₂, CO, H₂, mercury, and sulfur comprising contacting said fluid stream with a sorbent comprising metallic copper, wherein said sorbent is formed by combination of one or more copper oxysalts and alumina to produce a supported copper oxysalt and then metallic copper is formed from direct reduction of said supported copper oxysalt by exposure to a reducing agent at a temperature of between about 40° C. and about 220° C.

2. The method of claim 1, wherein said sorbent further comprises a reduction inhibitor and a copper oxide that are present both before and after the direct reduction.

3. The method of claim 2, wherein the reduction inhibitor is a halide salt.

4. The method of claim 3, wherein the reduction inhibitor comprises chloride ions.

5. The method of claim 2, wherein said copper oxide comprises cuprous oxide.

6. The method of claim 5, wherein said sorbent comprises no cupric oxide.

7. The method of claim 5, wherein said cuprous oxide is formed by direct reduction of said copper oxysalt without thermal decomposition to an oxide.

8. The method of claim 2, wherein said copper oxide comprises cupric oxide and cuprous oxide.

9. The method of claim 2, wherein said metallic copper comprises about 25 mass percent to about 50 mass percent, calculated as CuO on a volatile-free basis, of the copper-containing materials in said sorbent.

10. The method of claim 1, wherein said copper oxysalt is selected from the group consisting of basic copper carbonate and mixed copper carbonates.

11. The method of claim 1, wherein the copper oxysalt is Cu₂(OH)₂CO₃.

12. The method of claim 1, wherein said temperature is below a Hüttig temperature of said metallic copper.

13. The method of claim 1, wherein said sorbent further comprises a support material.

14. The method of claim 13, wherein said support material is selected from the group consisting of alumina, silica, silica-aluminas, silicates, aluminates, silico-aluminates, zeolites, titania, zirconia, hematite, ceria, magnesium oxide, and tungsten oxide.

15. The method of claim 1, wherein said reducing agent comprises at least one gas in the group consisting of hydrogen gas, carbon monoxide gas, synthesis gas, and hydrocarbon gas.

16. The method of claim 1, wherein said direct reduction occurs in an atmosphere comprising hydrogen at a partial pressure between about 10 bar (145 psi) and 120 bar (1740 psi) and a temperature between about 40° C. and about 105° C.
